(19) United States
(12) Patent Application Publication
(10) Pub. No.: US 2016/0271199 A1
(43) Pub. Date: Sep. 22, 2016

(54) Taste Modifiers and Therapeutic Methods Thereof

(71) Applicant: Alvin David Needleman, Las Vegas, NV (US)

(72) Inventor: Alvin David Needleman, Las Vegas, NV (US)

(73) Assignee: Weight Sciences, LLC, Las Vegas, NV (US)

(21) Appl. No.: 14/664,744

(22) Filed: Mar. 20, 2015

(51) Int. Cl. A61K 36/81; A61K 9/00; A61K 31/165; A61K 31/085; A61K 36/61

(52) U.S. Cl. CPC A61K 36/81; A61K 31/085; A61K 36/61; A61K 31/165; A61K 9/0056

(57) ABSTRACT

Taste modifier products and therapeutic methods for encouraging decrease in high calorie food intake by a person are disclosed. A taste modifier product ingested toy a person may comprise one or more sweetness inhibitors and one or more anesthetics. The taste modifier product may decrease sensitivity to sweet and bitter tastes when the product is ingested by the person. One or more coolant ingredients may also be included in a taste modifier product. The ingredients of a taste modifier product may be incorporated into a lozenge, a beverage, a chewing gum, an oral spray, an oral powder, a hard or a soft candy, a dessert or a foodstuff. When consumed by a person, the products may both depress a person's appetite and relieve hunger symptoms. Consequently, a person consuming the products may select smaller portion sizes when eating food and limit high calorie foods.

### FIELD OF THE INVENTION

This invention relates generally to sweetness inhibition and, more specifically, to proved taste modifier products Ingested by a person to encourage decrease in high calorie food intake by the person and therapeutic methods thereof.

### BACKGROUND OF THE INVENTION

Taste modification, and in particular sweetness inhibition, has been the subject of numerous studies. Receptor sites for sweetness are the subject of much research with an ever increasing understanding of where such receptor sites may occur. The identification of sweetness receptor sites is extremely difficult due to inadequate biochemical techniques appropriate for studying receptor binding in gustation (tasting).

Currently, it is believed that sweet taste response is mediated by taste cell surface receptors that utilize the adenylate cyclase system as a secondary messenger system. It is further believed that sweet taste receptors may be desensitized via a complex biochemical mechanism. Accordingly, various studies have been directed to understanding the structure versus activity relationship of various compositions on taste receptor sites.

Recently, a new model of the human sweet-taste receptor proposed by Temussi et al. J. Med. Chem. 2005, 48, 5520 may describe why different molecules taste sweet and why sweet tastes are additive. The concept of this new model is that taste receptors have different binding sites which mediate the perception of taste. One suggestion, of this model is that sweeteners can bind independently, which may explain how sweet taste can be additive. This model is currently being investigated both experimentally and by using modeling computations.

Various compositions axe known to modify taste perception. The mechanism for such taste perception alterations is not well understood. Complex formulations have been suggested that may provide taste modification. One objective of such formulations has been to reduce the desire for sweet-tasting high calorie foods. These formulations may result in a reduction of high calorie food intake. However, these formulations may provide side-effects which may make their use unpalatable for many people and lessen people's desire to use such formulations.

High calorie sweeteners such as sucrose are used in many foods for a variety of reasons. Public understanding of why high calorie sweeteners are used in foods is limited. A side-effect of use of high calorie sweeteners in food may be an increased desire for even higher calorie food intake.

Regulatory requirements dictate what ingredients may toe used in ingestible products. A commonly recognized standard in the U.S. is GRAS (generally recognized as safe).

The current disclosure provides GRAS compliant improved taste modifying products which are both satisfying and palatable. An advantage of the disclosed products is that they are therapeutically effective in reducing the desire for high calorie food sweeteners. A further advantage is that self-dosage with the disclosed products is simple to understand and apply.

According to one embodiment of the invention, a taste modifying lozenge is disclosed. The taste modifying lozenge may comprise in combination, at least one sweetness inhibitor, at least one anesthetic and at least one of a non-calorie sweetener and a low calorie sweetener. The combination of the at least one sweetness inhibitor, at least one anesthetic and at least one of a non-calorie sweetener and a low calorie sweetener may be configured to decrease a desire to consume both sweet and bitter food by a person after tasting the lozenge. The lozenge may further comprise at least one coolant ingredient. The coolant ingredient may be configured to modulate the perception of sweetness and bitterness toy the person.

According to another embodiment of the invention an ingestible taste modifier product is disclosed. The taste modifier product may comprise in combination, at least one sweetness inhibitor and at least one anesthetic. Both the at least one sweetness inhibitor and the at least one anesthetic may decrease sensitivity of the person to sweet and bitter tastes when ingested by the person. The taste modifier product may be selected from the group consisting of a lozenge, a beverage, a chewing gum, an oral spray, an oral powder, a hard candy, a soft candy, a dessert and a foodstuff.

The taste modifier product may further comprise at least one of a non-calorie sweetener and a low calorie sweetener. Furthermore, the taste modifier product may comprise at least one coolant ingredient configured to modulate the perception of sweetness and bitterness by the person. The at least one sweetness inhibitor may be selected from the group consisting of at least one gymnemic acid, hodulcin, ziziphin, an extract of Gymnema Sylvestre, an extract of Hovenia Dulcis and an extract of Ziziphus Jujuba. The at least one anesthetic may be selected from the group consisting of eugenic acid, an extract of Syzygium Aromaticum, sweet bell pepper, capsaicin and an antipyretic.

According to yet another embodiment of the invention a therapeutic method to encourage decrease in high calorie food intake by a person is disclosed. In one step the method comprises providing a taste modifying lozenge having at least one sweetness inhibitor, at least one anesthetic and at least one of a non-calorie sweetener and a low calorie sweetener. In another step the method comprises consuming the lozenge by the person thereby speeding onset of anesthesia of a portion of a tongue of the person. In yet another step the method comprises decreasing sensitivity of a portion of the tongue to sweetness and bitter tastes for at least 3.5 hours after tasting the lozenge. When the person ingests at least about 0.1 gram of the lozenge the person may both depress appetite and relieve hunger symptoms and select lower portion sizes of food to decrease high calorie food intake by the person.

The foregoing and other articles, features, and advantages of the invention will be apparent from the following more detailed description of the preferred embodiments of the invention. The various features may be utilized or claimed alone or in any combination.

### DESCRIPTION OF THE INVENTION

While it is well-known that sugars are present in many foodstuffs, it less well understood that sugars of various types have certain beneficial properties. Some beneficial properties of sugars may include acting as antimicrobials to extend shelf-life of foodstuffs, humectants to control water absorption and retention, fillers to increase bulk and rheology modifiers to control flow of ingredients making up a foodstuff during processing of the foodstuff.

In the context of this disclosure, sugars may comprise polysaccharides including sucrose, fructose, glucose, lactose, sugar alcohols, other carbohydrate sweeteners and the like. It will be appreciated that some complex polysaccharides are more easily metabolized to products available for absorption by a person's body, while other complex polysaccharides may require greater energy input from the body than the energy available from the products of metabolization.

Without limiting the scope of this disclosure, the components of the taste modifier products disclosed herein may reduce a person's desire to increase polysaccharide intake. The products may selectively block a person's taste receptors for high calorie sweet foods without inhibiting the sensation of taste for other non-sweet foodstuffs. Additionally, components of the products may be incorporated in one or more lozenges, beverages, chewing gums, oral sprays, oral powders, hard and soft candies, desserts and foodstuffs, and the like. Without limitation, an ingestible product comprising both the least one sweetness inhibitor and the at least one anesthetic may be configured to have any convenient physical state. An ingestible product may have any convenient physical state including one or more of the following: a semi-solid, a solid, a fluid, a suspension or an aerosol. Furthermore, the quantities of use and the components of the products may comply with the GRAS standard.

Without limiting the disclosure, the taste modifier products disclosed herein comprise active components effective in preventing a person's desire to ingest undesirable calorific sweets. These active components may comprise one or more sweet inhibitors and one or more anesthetics (or analgesics).

In one embodiment, a sweetness inhibitor of an exemplary taste modifier product comprises one or more extracts of Gymnema Sylvestre. The leaves of Gymnema sylvestre (GS) interfere with the ability to taste sweetness when chewed by a person, but also have a bitter taste. In the Hindi language, the leaves of GS are called gurmar. When translated from Hindi gurmar means destroyer of sugar.

Extracts of GS may include two resins with one of which is soluble in alcohol. Extracts of GS also may comprise gymnemic acids, saponins, stigmasterol, guercitol, and derivatives of amino acids including betaine, choline and trimethylamine.

Turning now specifically to extracts of the leaves of GS, studies indicate the following chemical components may be present: hentriacontane, pentatriacontane, alpha and beta chlorophylls, phytin, resins, tartaric acid, formic acid, butyric acid, anthraquinone derivatives, inositol, d-quercitol, gymnemic acid, alkaloids, oleanane-type triterpene glycosides, oleanane-type and tritepenoid saponins and O-iso-propylidene derivatives of gymnemagenin.

Gymnemic acid from the extract of GS leaves is thought to be a source of anti-sweetness and comprises a mixture of closely related acidic glycosides. These glycosides may also have a selective anesthetic effect both in topical applications and in competing with sweeteners for taste receptor sites.

Other examples of natural product extracts with similar anti-sweetness behavior similar to gymnemic acid include hodulcin (available from the leaves of Hovenia Dulcis) and ziziphin (available from the leaves of Ziziphus Jujuba). It is contemplated that any compounds or mixtures thereof providing anti-sweetness behavior that are safe for ingestion by humans may be suitable sweet inhibitors of the current disclosure.

In one embodiment, an anesthetic (analgesic) of an exemplary taste modifier product comprises one or more extracts of Syzygium Aromaticum (commonly known as cloves). Clove oil extracts from Syzygium Aromaticum comprise up to 95% eugenic acid and are considered to be GRAS. Clove oil extracts have a variety of uses including topical application in dentistry, as a flavoring in foods and beverages and in manufacturing of toothpastes, soaps, cosmetics and perfumes. When applied topically, eugenic acid is believed to depress sensory receptors in pain perception by inhibiting prostaglandin biosynthesis. It is contemplated that any compounds or mixtures thereof providing analgesic behavior that are safe for ingestion by humans may be suitable anesthetics of the current disclosure. Such compounds or mixtures may be natural or synthetic in origin and may include, without limitation extracts of sweet bell peppers such as disclosed by Belgorod in U.S. Pat. No. 6,086,888, capsaicins, antipyretics such as salicylates and the like.

These taste modifier products may comprise other active components including low calorie or non-calorie sweeteners configured to provide initial taste modification, thereby overwhelming a taste receptor's active sites. The sweeteners may be binders and provide bulk in appropriate ingestible taste modifier products.

In one embodiment of an exemplary taste modifier product, a non-calorie (or low calorie) sweetener may comprise one or more sugar alcohols including but not limited to erythritol, isomalt, maltitol, mannitol, sorbitol, xylitol and the like. Without limitation, other artificial sweeteners such as aspartame, saccharin, Acesulfame potassium, sucralose, D-tagatose and derivates thereof of any of these artificial sweeteners and the like may be suitable non-calorie or low calorie sweeteners of a contemplated taste modifier product.

Other active components of these taste modifier products may include ingestible coolants. Such coolants may promote temperature reduction of taste receptors thereby modulating the perception of sweetness and bitterness. While it is not known how coolants affect a taste receptor, cooling of taste receptor sites may influence binding to certain taste receptor sites. Some exemplary contemplated coolants may also have anesthetic properties as well.

In one embodiment of an exemplary taste modifier product, ingestible coolants may comprise one or more of menthol crystals and peppermint leaf powder and mixtures thereof. It is contemplated that both natural and synthetic coolants which are ingestible and GRAS may be suitable coolants of a taste modifier product. Suitable coolants may include, but are not limited to, dextro and levorotary isomers of menthol and menthyl derivatives. Examples of derivatized coolants may include menthyl lactate, menthone glycerol acetal, 3-1-menthoxypropane-1,2-diol, paramenthan carboxamides and the like. Other suitable coolants may comprise thymol, camphor, capsicum, eucalyptus oil, benzyl alcohol, salicylic alcohol, ethanol, spearmint oil, and mixtures thereof.

Additionally, excipients of various kinds may be used in these taste modifier products to control rheology (flow) and enhance binding capabilities of components. Such excipients may be both non-active and active components of formulations of these ingestible taste modifier products.

In one embodiment of an exemplary taste modifier product, excipients may comprise one or more of stearic acid, silicon dioxide, magnesium stearate, microcrystalline cellulose, mixtures thereof and the like. It is contemplated that a taste modifier product may comprise both natural and synthetic excipients and mixtures thereof. Suitable excipients may include one or more of starch and derivatives thereof such as sodium starch glycolate, pregelatinized starch. Other suitable excipients may include povidone and derivatives thereof such as crospovidone, cellulose and derivatives thereof such as hydroxy propyl methylcellulose, croscarmellose, hydroxy propyl cellulose, ethylcellulose, gelatin and derivates thereof, alkaline metal stearates such as calcium stearate, inorganic salts such as titanium dioxide, calcium phosphate and the like.

Other components of these taste modifier products may include colorants. It is contemplated that suitable colorants of a taste modifier product may comprise food grade dyes, pigments and mixtures thereof.

These colorants may provide psychological advantages to a person ingesting a taste modifier product. For example, blue colors may invoke a cooling sensation to a person, while red colors may invoke a greater desire to consume a lozenge. Colorants may further provide marketing advantage to a producer of such products. Additionally, colorants may be used as identifiers, product shelf-life indicators and production date indicators of specific taste modifier products. Identifiers and indicators of taste modifier products may further comprise indicia molded into any taste modifier products including but not limited to lozenges, soft candy, hard candy, chewing gum and the like.

One embodiment, of an exemplary taste modifier lozenge may comprise about 0.5 to about 15 parts by weight of an extract of Gymnema Sylvestre, and about 0.001 to about 0.020 parts by weight of an extract of Syzygium Aromaticum of a product comprising about 900 parts by weight of ingredients of the taste modifier lozenge. In another embodiment of a taste modifier product, about 0.05 percent to about 1.7 percent of a formulation of an ingestible taste modifier product comprises an extract of Gymnema Sylvestre and about 0.0001 percent to about 0.0025 percent of the formulation of the product comprises an extract of Syzygium Aromaticum. In yet another embodiment of an ingestible taste modifier product, about 0.03 percent to about 1.3 percent of a formulation of the product comprises one or more gymnemic acids and about 0.0001 to about 0.0025 percent of the formulation of the product comprises eugenic acid.

In yet another exemplary taste modifier lozenge may comprise about 89 percent of non-calorie (or low calorie) sweeteners including but not limited to one or more of xylitol, mannitol or sucralose. The taste modifier lozenge may further comprise about 2.2 percent of coolants including but not limited to one or more of menthol crystals or peppermint leaf powder. Furthermore, the taste modifier lozenge may comprise about 7.5 percent of recipients including but not limited to one or more of macrocrystalline cellulose, stearic acid, silicon dioxide or magnesium stearate.

### Exemplary Therapeutic Methods to Encourage Decrease of High Calorie Food Intake by a Person

In one embodiment of a method for encouraging decrease of high calorie food a person may be provided with a taste modifying lozenge having at least one sweetness inhibitor, at least one anesthetic and at least one of a non-calorie sweetener and a low calorie sweetener. The person may consume the lozenge and thereby speed onset of anesthesia of a portion of the person's tongue. A portion of the person's tongue may have decreased sensitivity to sweetness and bitter tastes for at least 3.5 hours after tasting the lozenge. Consequently, a desire for sweet or tart high calorie desserts may be limited by moderating or neutralizing normally perceived sweetness or tartness by a portion of a person's tongue.

After ingesting at least about 0.1 gram of the lozenge both the person's appetite may be depressed and hunger symptoms may be relieved. The person may select lower portion sizes of food, thereby lowering higher calorie food intake by the person.

In another embodiment of a method for encouraging decrease of high calorie food a person may be provided with a therapeutically effective amount of a taste modifying product of one or more sweetness inhibitors and one or more anesthetics. Both the one or more sweetness inhibitor and the one or more anesthetic may decrease sensitivity of a tongue of the person to sweet and bitter tastes when ingested by the person. At least one of the following: a losenge, a beverage, a chewing gum, an oral spray, an oral powder, a hard or a soft candy, a dessert or a foodstuff may comprise a portion of the product.

When the person consumes the taste modifying product, the sensitivity of the person's tongue to sweet and bitter tastes may be decreased. A portion of the person's tongue may have decreased sensitivity to sweetness and bitter tastes for at least 3.5 hours after tasting the product. By way of example without limiting the scope of the invention, a portion of 8 ounces of a beverage may be therapeutically active.

The person's appetite and desire for food may be decreased after ingesting the product. Consequently, the person may select smaller portion sizes of food and reduce their calorie intake. After the effects of the taste modifying product are diminished, the person may safely consume another therapeutically effective amount of the product, and continue to benefit from a decreased desire for eating sweet foods with high calorie content.

While the invention has been particularly shown and described with reference to preferred embodiments thereof, it will be understood by those skilled in the art that the foregoing and other changes in form and details may be made therein without departing from the spirit and scope of the invention. For example, ingredients of the taste modifier product may be provided in one or more ingestible products configured for consumption by a person. It is contemplated that the combination of the one or more ingestible products may provide the same benefit as a single ingestible product having the ingredients.

What is claimed is:

1. A taste modifying lozenge, comprising in combination:
at least one sweetness inhibitor, at least one anesthetic and at least one of a non-calorie sweetener and a low calorie sweetener, said combination configured to decrease a desire to consume both sweet and bitter food by a person after tasting said lozenge.

2. The lozenge according to claim 1 further comprising at least one coolant ingredient configured to modulate the perception of sweetness and bitterness by said person.

3. The lozenge according to claim 1 further comprising at least one excipient.

4. The lozenge according to claim 1 wherein about 0.05 percent to about 1.7 percent of said lozenge comprises an extract of Gymnema Sylvestre.

5. The lozenge according to claim 1 wherein about 0.0001 percent to about 0.0025 percent of said lozenge comprises an extract of Syzygium Aromaticum.

6. The lozenge according to claim 1 wherein about 0.03 percent to about 1.3 percent of said lozenge comprises at least one gymnemic acid.

7. The lozenge according to claim 1 wherein about 0.0001 percent to about 0.0025 percent of said lozenge comprises eugenic acid.

8. The lozenge according to claim 1 wherein about 89 percent of said lozenge comprises at least one of a non-calorie sweetener and a low calorie sweetener selected from the group consisting of xylitol, mannitol and sucralose.

9. The lozenge according to claim 2 wherein about 2.2 percent of said lozenge comprises at least one coolant ingredient selected from the group consisting of menthol crystals and peppermint leaf powder.

10. The lozenge according to claim 3 wherein about 7.5 percent of said lozenge comprises at least one excipient selected from the group consisting of microcrystalline cellulose, stearic acid, silicon dioxide and magnesium stearate.

11. An ingestible taste modifier product, comprising in combination;
at least one sweetness inhibitor and at least one anesthetic, both said at least one sweetness inhibitor and said at least one anesthetic decreasing sensitivity of said person to sweet and bitter tastes when ingested by said person.

12. The product according to claim 11 selected from the group consisting of a lozenge, a beverage, a chewing gum, an oral spray, an oral powder, a hard candy, a soft candy, a dessert and a foodstuff.

13. The product according to claim 11 further comprising at least one of a non-calorie sweetener and a low calorie sweetener.

14. The product according to claim 11 further comprising at least one coolant ingredient configured to modulate the perception of sweetness and bitterness by said person.

15. The product according to claim 11 further comprising at least one excipient.

16. The product according to claim 11 comprising at least one sweetness inhibitor selected from the group consisting of at least one gymnemic acid, hodulcin, ziziphin, an extract of Gymnema Sylvestre, an extract of Hovenia Dulcis and an extract of Ziziphus Jujuba.

17. The product according to claim 11 comprising at least one anesthetic selected from the group consisting of eugenic acid, an extract of Syzygium Aromaticum, sweet bell pepper, capsaicin and an antipyretic.

18. The product according to claim 13 comprising at least one of a non-calorie sweetener and a low calorie sweetener selected from the group consisting of erythritol, isomalt, maltitol, mannitol, sorbitol, xylitol, apartame, aspartame derivates, saccharin, Acesulfame potassium, sucralose, and D-tagatose.

19. The product according to claim 14 comprising at least one coolant ingredient selected from the group consisting of at least one of dextro and levorotary isomers of menthol, menthone and menthyl derivatives, camphor, capsicum, eucalyptus oil, benzyl alcohol, salicylic alcohol, ethanol, spearmint oil, menthol, crystals and peppermint leaf powder.

20. A therapeutic method to encourage decrease in high calorie food intake by a person comprising the steps of:
providing a taste modifying lozenge having at least one sweetness inhibitor, at least one anesthetic and at least one of a non-calorie sweetener and a low calorie sweetener;
consuming said lozenge by said person thereby speeding onset of anesthesia of a portion of a tongue of said person; and
decreasing sensitivity of a portion of said tongue to sweetness and bitter tastes for at least 3.5 hours after consuming said lozenge.

21. The therapeutic method of claim 20 further comprising the steps of:
ingesting at least about 0.1 gram of said lozenge by said person to both depress appetite and to relieve hunger symptoms of said person; and
selecting lower portion sizes of food, thereby decreasing higher calorie food intake by said person.
